(19) United States
(12) Patent Application Publication
(10) Pub. No.: US 2009/0216149 A1
(43) Pub. Date: Aug. 27, 2009

(54) SELF-CONTAINED, IMPLANTABLE, INTRACRANIAL PRESSURE SENSING DEVICE AND METHODS FOR ITS USE IN MONITORING INTRACRANIAL PRESSURE

(75) Inventors: Samuel R. Neff, Ardmore, PA (US); Janine Neff, Ardmore, PA (US); Arye Rosen, Cherry Hill, NJ (US); Mohammad-Reza Tofighi, Malvern, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: DREXEL UNIVERSITY, Philadelphia, PA (US)

(21) Appl. No.: 12/094,875

(22) PCT Filed: Dec. 1, 2006
(86) PCT No.: PCT/US06/61451

§ 371 Date: Nov. 6, 2008

Related U.S. Application Data
(60) Provisional application No. 60/741,308, filed on Dec. 1, 2005.

(51) Int. Cl. A61B 5/03

(52) U.S. Cl. 600/561

(57) ABSTRACT

A reliable and mass-producible microelectromechanical (MEMS)-based microwave intracranial pressure sensing device for use with a portable microwave monitor and methods for non-invasively monitoring intracranial pressure with this device are provided.

This patent application claims the benefit of priority from U.S. Provisional Application Ser. No. 60/741,308, filed Dec. 1, 2005, teachings of which are herein incorporated by reference in their entirety.

This invention was supported in part by funds from the U.S. government (NIH Grant No. 1 R21 NS050590-01). The U.S. government may therefore have certain rights in the invention.

### FIELD OF THE INVENTION

The present invention relates to a reliable and mass-producible microelectromechanical (MEMS)-based microwave intracranial pressure (ICP) sensing device which, when used with a portable microwave monitor, allows for non-invasive monitoring of ICP.

### BACKGROUND OF THE INVENTION

Head injuries and diseases of the brain are major causes of death and disability in the developed nations. Stroke is the third leading cause of death in the United States, and head injury is a leading cause of death in adolescents and young adults. Hydrocephalus accounts for over 50,000 hospital admissions each year. Between 5,000 and 15,000 people receive a new diagnosis of intracranial tumor, 100,000 have a hemorrhagic stroke, and 1.5 million have a traumatic brain injury. Clinical determination of intracranial pressure is critical to the management of each of these conditions.

Intracranial pressure ranges from approximately −5 to 10 torr in the normal human. Since the skull forms an almost complete rigid container for the brain, measuring intracranial pressure directly is not possible. However, penetration of the skull to insert a pressure sensor requires a neurosurgical procedure with significant risks. Thus, measuring intracranial pressure remotely is preferable.

Existing neurosurgical intracranial pressure (ICP) monitors can only be used in the hospital setting, and have limited useful life due to drift and infection.

A number of neurocranial monitors have been described that are purported to facilitate measurement of intracranial pressure. These devices can be grouped into four main categories, namely devices with radiofrequency tuned circuits, devices with vibrating mechanical components, devices with moving magnetic components, and devices with optical components.

However, devices with significant inductive or magnetic components, including all radiofrequency circuit-based devices are not compatible with magnetic resonance imaging, a procedure often critical to management of patients with abnormal intracranial pressure. Further, many of these devices have a limited lifetime, particularly devices with plastic components, which age rapidly in vivo when in contact with extracellular space, or slide bearings, which are not reliable over long term. The accuracy of these devices can also be degraded by scar formation and/or requirement for a cerebrospinal fluid (CSF) path. For example, a device relying on measurement via a flexible diaphragm will be useless if encased in relatively stiffer scar tissue while a device requiring CSF flow will be prone to clogging in more than 60% of patients based on current CSF shunt data. In addition, many of these devices require either a large number of parts, precise machining or rare and/or exotic materials making manufacture and assembly cost prohibitive.

Passive (battery-less) Bio-MEMS pressure sensors operating at 10-20 MHz (DeHennis, A. and Wise, K. D. Digest of IEEE Conference on MicroElectroMechanical Systems 2002 252-255) and 330 MHz (Simons et al. Digest of 2004 IEEE International Microwave Symposium 2004 3:1433-1436) have been described. These sensors require transcutaneous inductive links for monitoring of the pressure. Since these inductive links operate at near-field, the pressure monitoring receiver must be placed on the surface of the body. Accordingly, no remote monitoring is possible. Further, these implants require large inductors (e.g. 3.7 μH (DeHennis, A. and Wise, K. D. Digest of IEEE Conference on MicroElectroMechanical Systems 2002 252-255) and 150-200 nH (Simons et al. Digest of 2004 IEEE International Microwave Symposium 2004 3:1433-1436), which are not compatible with magnetic resonance imaging.

Accordingly, there is a need for a stable, biocompatible, rugged and inexpensive intracranial pressure sensor sufficiently small to be inserted through the burr hole and left inside the cranium following most common neurological procedures which is compatible with modern imaging techniques including, but not limited to CT, MRI and ultrasound and which monitors intracranial pressure via a simple, rapid and painless interrogation procedure.

### SUMMARY OF THE INVENTION

An object of the present invention is to provide a reliable and mass producible MEMS-based microwave ICP device sized for implantation into the cranium through a burr hole during a neurosurgical procedure. The device comprises a chip with an oscillator and an oscillator bias control circuit, a microwave antenna coupled to the oscillator output, a sensing component, preferably an MEMS capacitor, whose variation with the intracranial pressure changes the oscillation frequency of the oscillator, and a power source.

Another object of the present invention is to provide with this intracranial pressure measuring device comprising this reliable and mass-producible MEMS-based microwave ICP sensing component, a portable microwave monitor for display and external monitoring of the oscillator output transmitted via the antenna of the device and received by this portable microwave monitor.

Another object of the present invention is to provide methods for monitoring intracranial pressure in a subject via these devices.

### BRIEF DESCRIPTION OF THE FIGURES

FIG. 1 is a side view of a diagram of one embodiment of an implantable sensing device of the present invention.

FIG. 2 is a top view of the same implantable sensing device of the present invention depicted in FIG. 1.

FIG. 3 is a diagram showing the position of the sensing device following surgical implantation in the cranium.

FIG. 4 is a schematic of an exemplary LC CMOS oscillator.

### DETAILED DESCRIPTION OF THE INVENTION

The final common pathway for death and permanent disability in head injuries and brain diseases is usually increased intracranial pressure. For this reason, measurement and control of intracranial pressure is a major focus of care of these individuals, both acutely and chronically. Since the intracranial contents exist within the rigid confines of the skull, direct pressure measurements require neurosurgical procedures, with their attendant risks, discomforts, and expense. Existing neurosurgical intracranial monitors can only be used in the hospital setting, most typically in the intensive care unit, and have limited useful life due to drift and infection. Indirect inferences about intracranial pressure are often made from neuroimaging studies, but these can be misleading, sometimes fatally so. Neuroimaging studies are also expensive and may not be convenient. Certainly neuroimaging is not a realistic option for an outpatient with chronic symptoms.

However, the patients whom knowledge of intracranial pressure is most critical are those patients having undergone an intracranial invasive procedure.

In the present invention an intracranial pressure measuring device is provided that can be implanted inside the cranium during a neurosurgical procedure. The device of the present invention preferably comprises a reliable and mass-producible MEMS-based microwave ICP sensing component and a portable microwave monitor and provides for non invasive monitoring of ICP.

More specifically, the intracranial pressure measuring device of the present invention comprises an oscillator-based surgically implantable unit operating at microwave frequencies which measures intracranial pressure. A unit operating at microwave frequencies was selected for various reasons including its frequency sensitivity to the change in its tank capacitor and the ability to detect the microwave signal transmitted by a small antenna inside the implant from a significant distance outside the patient. An ISM band microwave frequency of 2.4 GHz is high enough to be efficiently radiated by a small antenna, but is low enough to avoid significant absorption by the implant package and scalp.

Sensing components, electronics, and antenna are assembled on printed circuit boards constructed of silicon dioxide or aluminum oxide substrate. For biocompatibility, the device is coated with a very thin layer of Parylene (polymerized para-xylylene).

In a preferred embodiment the sensing component comprises an oscillator operating at the Industrial-Scientific-Medical (ISM) band of 2.4000-2.4835 GHz. In this embodiment, the sensing component preferably comprises a tank oscillator with LC components selected to confine the range of oscillation to 2.4000 to 2.4835 GHz for the pressure range of −25 to 200 torr, which corresponds to about S=0.37 MHz/torr sensitivity. It is preferred that a CMOS chip, fabricated by a submicron CMOS process, including the oscillator and an oscillator bias control circuit be used since CMOS is a commercially available, low-power consuming technology. Further, the CMOS oscillator is based on a differential cross-coupled topology (Razavi, B. in Design of Integrated Circuits for Optical Communications, New York, McGraw Hill 2002) and thus has the advantage of requiring four times lower bias current for oscillation compared to the traditional Colpitts oscillator.

A schematic of an exemplary LC CMOS oscillator useful in the sensing component of the present invention is depicted in FIG. 4. This LC CMOS oscillator is also described in detail by Razavi, B. in Design of Integrated Circuits for Optical Communications, New York, McGraw Hill 2002. In this depicted embodiment the intracranial pressure sensing component constitutes the capacitor of the tank circuit (C) and its capacitance change directly changes the oscillation frequency. Tank inductor L and an additional capacitor C₀ are selected to confine the range of oscillation from 2.4000 to 2.4835 GHz, for the pressure range of −25 to 200 torr, which corresponds to about S=0.37 MHz/torr sensitivity with the pressure change. For a capacitor variation of 1.3 to 3.5 pF, simulation yields a 2.40 to 2.48 GHz oscillation, where values of L=22.9 nH and C₀=0.17 pF are also selected. Output power is fairly constant and is about −7.58 dBm (0.17 mW). Total DC current and consumed power is 11.5 mA and 34 mW.

The CMOS chip also preferably comprises a bias control circuit such as a CMOS timer to save battery power. This control circuit provides a means for the oscillator bias to be switched on and off periodically. For example, in one embodiment, the oscillator bias is switched on and off periodically, with a period of T=100 ms, and a pulse width of T₀=10 μs. This period of 100 ms can be generated by a three stage ring oscillator such as described by Razavi, B. in Design of Integrated Circuits for Optical Communications, New York, McGraw Hill 2002. The 10 μs microwave pulse has a bandwidth of 1/10 μs=0.1 MHz, which corresponds to a pressure resolution of 0.27 torr (±0.135 torr) for S=0.37 MHz/torr. In this embodiment, the duty cycle of T₀/T=0.0001 corresponds to an average current of 1.1 μA for the microwave oscillator. This is considerably lower than the rest of the CMOS circuitry which typically operates at approximately 20 μA (Stotts, L. J. IEEE Circuits and Devices Magazine 1989 5(1):12-18). Based upon data in pacemakers, it is expected that the low average oscillator current would allow a lifetime of about 2 months for a 30 mA/h battery.

The device further comprises a sensing component, preferably a MEMS capacitor, whose variation with the intracranial pressure changes the oscillation frequency of the oscillator. An alternative to the MEMS capacitor sensing component is a piezoresistive pressure sensor. In this alternative approach, the piezoresistive sensor output is applied through signal conditioning circuitry (instrumentation amplifier) to the tuning voltage of a voltage controlled oscillator, while the rest of the electronics is the same as the electronics for the capacitive MEMS based device. However, the device based on MEMS capacitor is preferred for most embodiments as it offers advantages such as consuming less power, being more compact by not having the signal conditioning circuitry, and having a pressure monitoring output (frequency change with ICP change) less sensitive to the battery voltage and temperature changes.

The oscillator output is coupled to an antenna which transmits the output to an external monitoring and/or display unit. An example of an antenna useful in the device of the present invention is the 2.4 GHz Bluetooth chip antenna 2.2 mm×6.5 mm² in size (LINX Technology). This type of antenna is fabricated on a very high dielectric constant substrate and can be easily mounted on a printed circuit board as a surface mount component. This exemplary antenna has an input impedance of 50Ω and 3 dB bandwidth of 180 MHz.

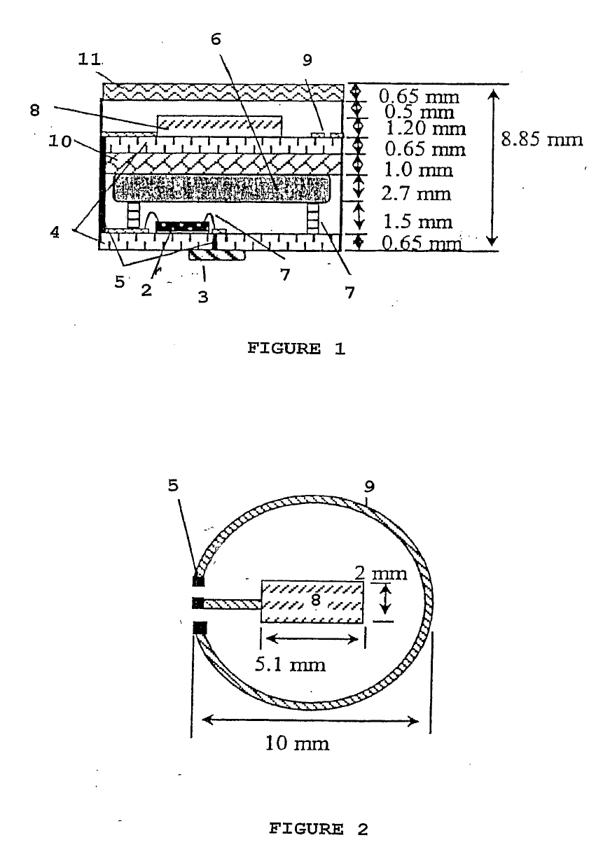

Power to the device of the present invention is preferably provided via a small rechargeable battery such as a 3 V, 30 mAh capacity, lithium battery. Total DC current and consumed power of the exemplary device depicted in FIGS. 1 and 2 is 11.5 mA and 34 mW. Batteries such as these can be recharged by external means such as optically via a laser generated current and a photovoltaic diode. A photovoltaic diode illuminated by an 870 nm laser beam was demonstrated to generate a voltage of 0.4 V at 47 mW. Alternatively, the battery can be recharged via an inductive link which requires placement of a planar coil surrounding the antenna. See FIG. 1.

The device of the present invention is sized sufficiently small so that is can be implanted through a burr hole typically 12 mm in diameter. An exemplary embodiment of a device of the present invention is depicted in FIGS. 1 and 2. In this exemplary embodiment, a CMOS chip 2 and sensing component 3 are mounted at two sides of an alumina substrate 4. The sensing component 3 is connected to the chip 2 through vias 5. Tank inductance is implemented inside the chip. In this exemplary embodiment, the battery 6 is on top of the chip separated by wirebond supports 7. A second alumina substrate 4 separated from the battery by a spacer 10 contains an antenna 8 and a means 9 such as a printed circuit spiral inductor or photodiode array for recharging the battery. These components are also wired to the CMOS chip 2 through the vias 5 at the left side of FIGS. 1 and 2. This device takes up a cylindrical volume of 10 mm in diameter and 8.85 mm in height. As will be understood by the skilled artisan upon reading this disclosure, alternative arrangement of the components of the exemplary device depicted in FIGS. 1 and 2 can be used. Further, the printed circuit substrates, on which the sensing component, electronics, and antenna are placed, can have lower thickness (as thin as 0.15 mm). Also air spaces in the device depicted in FIG. 1 can be reduced (or increased) to provide devices in the range of 5 to 10 mm in height.

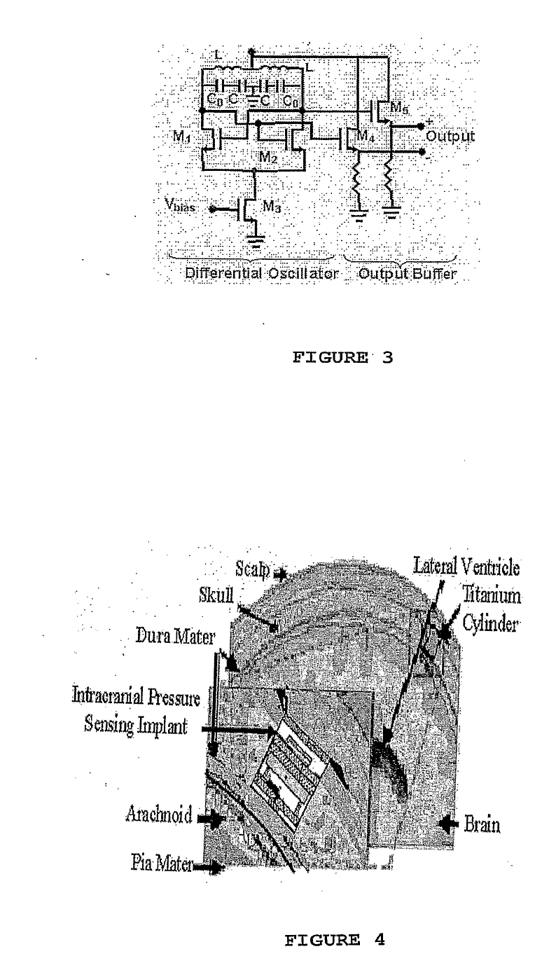

The device depicted in FIGS. 1 and 2 is packaged within a titanium cylinder for ruggedness and biocompatibility. The Teflon window 11 in FIG. 1 is a microwave transparent layer which seals the antenna and electronics from the scalp. In an alternative embodiment, the Teflon is replaced with a thin layer (0.1-0.2 mm) of a biomedical grade silicone sealant. The device packaged in the titanium case and sealed is further coated with a layer of Parylene over all exterior surfaces, including the surface of the sensing component. As shown in FIG. 3, the surface of the sensing component is in contact with or exposed to the dura mater. Accordingly, the Parylene coating must be sufficiently thin, preferably about 2.5 μm thick, so that it has no impact on the sensing component's sensitivity or other characteristics. No layer of silicone sealant or Teflon is placed between the surface of the sensing component and dura mater. Instead, the silicone sealant or Teflon is applied only to areas of the device away from the sensing component's surface as a thin layer of sealant to prevent fluids and tissues from getting into the internal electronics of the device.

The intracranial pressure measuring device of the present invention may further comprise a monitor which detects signal irradiated by the antenna. In a preferred embodiment, the main component of the monitor is an ISM band zero-IF module such as that available from Maxim, Inc. However, as will be understood by the skilled artisan upon reading this disclosure, alternative microwave monitors, preferably simple, portable microwave monitors can be used. In this preferred embodiment, the sensitivity of the microwave monitor is −85 dBm or better.

The intracranial pressure measuring device of the present invention provides a useful means for post-operative long term monitoring of intracranial pressure in patients in need thereof. Use of CMOS technology in this device provides for relatively low cost in its production as well as low power usage. Further, the device of the present invention is compatible with most imaging techniques. In addition, the small size of the device coupled with external monitoring provides for a patient friendly means for monitoring intracranial pressure. The devices of the present invention are particularly useful in patients with hydrocephalus and intracranial tumors wherein long term monitoring of ICP is required.

The following nonlimiting example is provided to further illustrate the present invention.

### EXAMPLE

### Example 1

### Estimation of Skin Absorption

Experiments were performed with the device depicted in FIGS. 1 and 2 to estimate skin absorption (M.-R. Tofighi, U. Kawoos, S. Neff, and A. Rosen, Wireless Interacranial Pressure Monitoring Through Scalp at Microwave Frequencies, Submitted to Electronics Letters). Alternatively, the absorption and transmission of a plane wave through a Teflon window followed by a layer of skin, 1 or 2 mm thick, with known complex permittivity (Gabriel et al. Phys. Med. Biol. 1996 41:2271-2293) was evaluated analytically. The absorption was lower than 20% of the incident power at 2.45 GHz. In addition, the transmission was about 18 to 43%. Assuming that the pose was absorbed over a cylindrical volume of skin with 10 mm diameter, SAR=0.2-0.4 W/kg was expected. This SAR is sufficiently below the 1.6 W/kg of the ANSI/IEEE RF safety guidelines. Moreover, given the duty cycle of 0.0001 for signal transmission, the actual average SAR is four orders of magnitude lower than the above.

1. An intracranial pressure device comprising a chip with an active microwave oscillator and an oscillator bias control circuit, a microwave antenna coupled to an output of said active microwave oscillator, a sensing component whose variation with the intracranial pressure changes the microwave oscillation frequency of the oscillator, and a power source, said device being sized for implantation into the cranium through a burr hole during a neurosurgical procedure and wherein said device transmits said changes in oscillation frequency via said microwave antenna.

2. The device of claim 1 wherein the sensing component comprises a microelectromechanical (MEMS)-based capacitor.

3. The device of claim 1 further comprising a portable microwave monitor for display and external monitoring of oscillator output transmitted via the antenna of the device and received by this portable microwave monitor.

4. A method for monitoring intracranial pressure in a subject comprising implanting the device of claim 1 into the cranium of a subject via a burr hole and monitoring intracranial pressure externally via a portable microwave monitor.
